(19) United States
(12) Patent Application Publication
(10) Pub. No.: US 2009/0156910 A1
(43) Pub. Date: Jun. 18, 2009

(54) Noninvasive glucose monitor and method

(76) Inventors: Yanping Kong, Newton, MA (US); Youchun Kong, Newton, MA (US)

Correspondence Address: WILLIAM NITKIN, 1320 CENTRE STREET, SUITE 300, NEWTON, MA, 02459, US

(21) Appl. No.: 12/002,263

(22) Filed: Dec. 17, 2007

(51) Int. Cl. A61B 5/00; A61B 8/00

(52) U.S. Cl. 600/306; 600/347

(57) ABSTRACT

An instrument and method for determining the current blood glucose level of an individual by noninvasive measurement of the current electrical capacitance of the outer surface of the skin of such individual and comparing that current capacitance level to stored data of previously determined capacitance levels of the skin and their associated blood glucose levels of that individual for determining the blood glucose level associated with the currently determined capacitance level.

### BACKGROUND OF THE INVENTION

1. Field of the Invention

The device and method of this invention reside in the area of blood glucose monitors for use by diabetic individuals and more particularly relate to a noninvasive blood glucose monitor and method for such individuals to determine their blood glucose levels using electrical capacitance readings obtained from their skin.

2. Background of the Prior Art

There are approximately 21 million individuals with diabetes mellitus in the United States. These individuals must monitor their blood glucose levels as part of their medical care. It is well known that individuals who more diligently monitor their blood glucose levels can have a better prognosis. In one method of invasive blood glucose monitoring the individual pricks his finger with a lancet to obtain a drop of blood which is manually placed on a test strip which, in some embodiments, can change color to indicate the blood glucose level or in other embodiments can be read by a monitor which automatically displays numerically the blood glucose level on the screen of the monitor. Another method uses sensors implanted under the skin. Finger pricking can be painful and uncomfortable for some individuals, many of whom do not tolerate it well. The pain and inconvenience associated with such finger pricking causes a large number of individuals to test their blood glucose levels less frequently and therefore not monitor their blood glucose levels as often as recommended.

There are at present several new techniques for noninvasive blood glucose monitoring which involve, in one instance, the exterior measurement of blood glucose levels using infrared waves and other types of spectral wave monitoring. These devices and methods are complicated and can be more costly than current invasive blood glucose testing methods. One of these noninvasive devices utilizes an excitation laser beam directed into the anterior chamber of an individual's eye so that the aqueous humor filling the anterior chamber of the eye is illuminated. The device detects a spectrum from the illuminated aqueous humor from which the blood glucose level can be determined. Other devices and methods utilize near infrared radiation which can pass into the eye and the reflected radiation is detected by a near infrared sensor which measures the reflected energy in one or more wavelength bands. The spectral absorption of the eye is measured and used to determine the level of blood glucose in the eye. Devices and methods requiring shining lights in the eye, though, are difficult tasks for individuals to accomplish by themselves. Other devices and methods have used the direction of infrared radiation onto the skin and on other areas of the body, such as the tympanic membrane, to determine the spectral emissivity of the infrared radiation from which the blood glucose concentration can be then determined.

### SUMMARY OF THE INVENTION

It is an object of this invention to utilize the measurement of an individual's electrical capacitance on the skin as a new means for determining that individual's blood glucose level.

It is a further object of this invention to provide a noninvasive monitor and method to determine blood glucose levels that is extremely reliable, safe and very convenient for individuals to use.

It is a still further object of this invention to substantially avoid the invasive testing of blood glucose levels such as by the pricking of the finger or other body parts to obtain blood for testing.

The body of a hypothetical individual when considered as a whole can be likened to a large bag of fluid with blood in it. If one further considers that bag as an electrical device, then its electrical capacitance at its surface can be measured. Changes in blood glucose levels change the electrical capacitance of an individual, as described below. Thus the changes in electrical capacitance of an individual before and after eating can be determined since changes in the blood glucose level change the individual's electrical capacitance. With an increase in the amount of electrical particles caused by an increase of glucose particles, the body's electrical capacitance is changed; and such electrical capacitance can be measured and correlated to determine the blood's glucose level at such times. Thus by measuring the electrical capacitance of an individual at various times during the day, one can accurately determine the individual's glucose level at those times. It should be noted that such hypothetical individual, being likened to a bag of fluid, contains electrically active particles such as particles of sodium, potassium, chloride, bicarbonate, blood urea nitrogen (BUN) and glucose. Except for glucose, all of these electrically active particles before and after such individual has eaten do not significantly change. The different amounts of particles of glucose in the individual at different times causes the individual to have a different capacitance which can be measured, such as before and after eating. By initially measuring the capacitance before and then after eating, one can determine the amount of particle change in the blood glucose level. It has been found that using a meter of 200 n differential ability, one can measure the capacitance of the human body by placing two electrical lead contacts on the skin, for example on the forearm. The monitor of this invention utilizes such a meter. Such monitor can simulate the appearance of a wrist watch which can be worn at the wrist or near the wrist, and the monitor can have two points of electrode contact thereunder which touch the individual's skin. One can use the monitor of this invention to measure the capacitance of the body and determine the blood glucose level therefrom.

### BRIEF DESCRIPTION OF THE DRAWING

FIG. 1 illustrates a perspective view of the noninvasive blood glucose monitor of this invention worn on the forearm of an individual.

FIG. 2 illustrates a chart that records the results of three actual blood glucose tests on an individual, showing an increase in electrical capacitance with an increase in blood glucose levels.

FIG. 3 illustrates a second chart that records the results of three actual blood glucose tests on a second individual, showing an increase in electrical capacitance with an increase in blood glucose levels.

### DESCRIPTION OF THE PREFERRED EMBODIMENT(S)

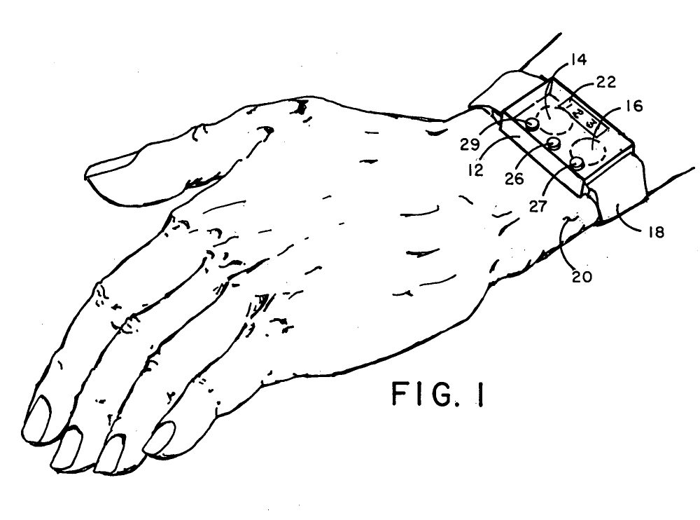

FIG. 1 illustrates a perspective view of the noninvasive blood glucose monitor of this invention worn on the forearm of individual 10. Blood glucose monitor 12 in a preferred embodiment is similar in size and appearance to a wrist watch. On its bottom outer surface are disposed a pair of first and second lead contacts 14 and 16. The monitor is held to the individual's arm by strap or band 18 whereby first and second lead contacts 14 and 16 make contact with skin 20 to determine the electrical capacitance of the individual's skin between first lead contact 14 and second lead contact 16. Monitor 12 can be activated by depressing “on” button 26 and the current blood glucose level as determined by the calculator using the stored electrical capacitance data relative to the blood glucose level can be displayed on screen 22 located on the front surface of the monitor. Although the monitor is shown in FIG. 1 as being rectangular, it can have other shapes as well.

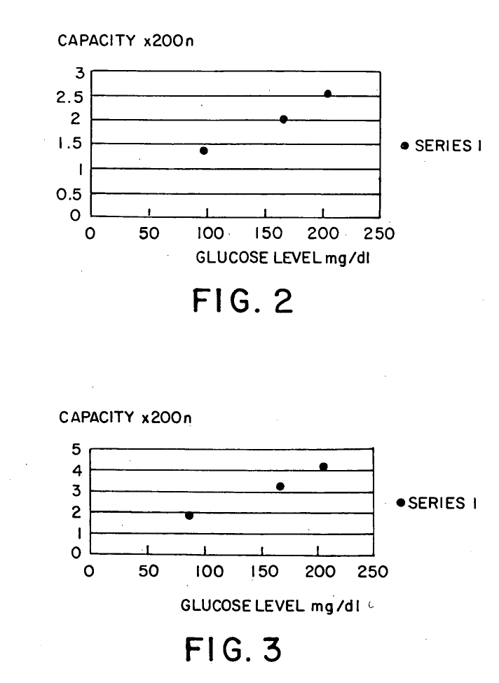

FIG. 2 illustrates a chart showing the results of three actual blood glucose tests and associated electrical capacitance on one individual, and FIG. 3 illustrates a chart showing the results of a three actual blood glucose tests and associated electrical capacitance on a second individual. The results of such tests show a significant correlation between an individual's blood glucose levels and the electrical capacitance of the individual's skin, showing that an increase in the blood glucose causes an increase in the skin's electrical capacitance.

In practice, to initially set the monitor of this invention, one measures both the capacitance values and associated blood glucose readings using a prior art finger prick glucose test for three or more times. For example:

| | | | |
|---|---|---|---|
| Blood Glucose Level (mg/dl) | 80 | 140 | 200 |
| Capacitance Level (n) | 2.3 | 3.2 | 5.1 |

The values should be the real values measured for the individual who is going to be using the monitor. For example, when the individual's blood glucose level is 80 mg/dl, that person's electrical capacitance is measured at 2.3 n. The monitor has three buttons on its front surface, being first button 26, second button 27 and third button 29. The values of the blood glucose levels can be entered each time through these buttons. Such digital entering of data into devices is well known. Each capacitance value within the monitor's calculator can be used to obtain a blood glucose level by the following formula:

Y=KX+B

where Y equals blood-glucose concentration level, K equals the slope of the calibration equation, X equals the capacitance measurement obtained by the monitor, and B equals the Y-axis intercept. After the various values have been entered, the computer in the monitor automatically calculates the K value and the B value of the formula and stores these values in its calculator system. Whenever the monitor detects an electrical capacitance value of the individual, it can then calculate the individual's blood glucose level and show the current blood glucose level on display screen 16. The monitor thus associates the current capacitance value to a particular blood glucose level and displays such blood glucose level information.

In a preferred embodiment the monitor of this invention can be similar in shape and size to a wrist watch and have four major components. The first component is a capacitance meter that can measure electric capacitance at 200 n to measure the electric capacitance of the skin on which the monitor is placed. The second component is a miniature computer which is in the form of a preset calculator that can compute the current measured capacitance value and determine, based on its stored data, the current blood glucose level. The third component consists of two electrical lead contacts disposed on the bottom outer surface of the monitor which lead contacts contact the skin, sense the individual's capacitance and send signals to the capacitance meter to determine present electric capacitance level of the skin. The fourth component is a battery providing electrical current to components of the monitor. The miniature computer which can be placed into the watch-size monitor of this invention can be preset as a calculator to calculate current blood glucose levels from the current capacitance values received from the capacitance meter and calculated according to the formula Y=KX+B, as discussed above.

The two lead contacts on the skin can have in one embodiment small, sharp heads which can be 1 mm in diameter and extend 2 mm out from the outer surface of the bottom of the monitor which outer surface should be made of plastic or other nonconductive material. Thus the monitor of this invention measures blood glucose levels on a noninvasive basis which can be simply obtained because it determines the blood glucose level based on the electrical capacitance of the outer surface of the skin only. The use of such monitor is non-painful, and the monitor can be worn in a manner similar to the wearing of a real wrist watch. When an individual needs to check his blood glucose level, he activates the watch-like monitor by pushing an “on” button which connects the battery to the other electrical components of the monitor. The monitor will then determine the real-time blood glucose level and display it on a screen on the face of the monitor. The monitor in a watch-like form, as shown in FIG. 1, can include other watch features, for example, a time display on the screen which can be interchanged with the blood glucose reading when desired.

It has been found that the use of the blood glucose monitor of this invention utilizing the measurement of electrical capacitance of the skin to obtain blood glucose readings is far superior to current complex infrared wave-reading systems for individuals to use and is far more desirable than the skin pricking methods of the prior art which discourage multiple blood glucose testing because of the pain involved in such testing.

Although the present invention has been described with reference to particular embodiments, it will be apparent to those skilled in the art that variations and modifications can be substituted therefor without departing from the principles and spirit of the invention.

We claim:

1. A method for determining the current blood glucose level of an individual having skin, said skin having an outer surface, comprising the steps of:
measuring by noninvasive means the current capacitance level of said outer surface of said skin of said individual;
comparing said current capacitance level to predetermined capacitance levels at various blood glucose levels;
determining the current blood glucose level of said individual by such comparison; and
displaying said current blood glucose level for observation by said individual.

2. The method of claim 1 further including the step of:
providing a monitor having a display for determining said current capacitance level of said individual, said monitor to be worn by said individual, said monitor having a top and bottom.

3. The method of claim 2 further including the steps of:
providing said monitor with a strap, said strap for strapping said monitor onto the wrist of said individual;
providing a pair of lead contacts on said bottom of said monitor; and
contacting said skin of said individual with said pair of lead contacts for determining said current capacitance level of said individual.

4. A method for determining the current blood glucose level of an individual having skin, said skin having an outer surface, comprising the steps of:
measuring a series of blood glucose levels of said individual;
measuring by noninvasive means the capacitance level of said outer surface of said skin of said individual at the same time as each blood glucose level is measured, each capacitance level having an associated blood glucose level;
storing the results of said capacitance levels and their associated blood glucose levels-of said individual;
measuring a current capacitance level of said outer surface of said skin of said individual; and
comparing said current capacitance level with said stored capacitance levels and their associated blood glucose levels to determine the current blood glucose level of said individual.

5. The method of claim 4 further including the step of:
providing a monitor having a display for determining said current capacitance level of said individual, said monitor to be worn by said individual, said monitor having a top and bottom.

6. The method of claim 5 further including the steps of:
providing said monitor with a strap, said strap for strapping said monitor onto the wrist of said individual;
providing a pair of lead contacts on said bottom of said monitor; and
contacting said skin of said individual with said pair of lead contacts for determining said current capacitance level of said individual.

7. An instrument for determining the current blood glucose level of an individual having skin, said skin having an outer surface, comprising:
means for detecting the current capacitance level of said individual at a point on said outer surface of said skin of said individual; and
means for comparing said current capacitance level to previously determined capacitance levels and their associated blood glucose levels of said individual for determining the blood glucose level associated with said current capacitance level.

8. The instrument of claim 7 wherein said instrument is in the form of a watch-like device held to the wrist of said individual by a watchband, said watch-like device having a top surface and a bottom surface, said current capacitance level detection means disposed on said bottom surface of said watch-like device, said watch-like device having display means disposed on said top surface for displaying the current blood glucose level of said individual.

9. The instrument of claim 8 wherein said current capacitance level detection means comprises a pair of lead contacts for contacting said skin of said individual.
